SEQUENCE LISTING (24 sequences)

SEQ ID NO: 1 - DNA, 315 bp, artificial sequence
  the seventh exon of porcine CD163 gene
cccacaggaaacccaggctggttggaggggacattccctgctctggtcgtgttgaagtacaacatggagacacgtggggcaccgtctgtgattctgacttctctctggaggcggccagcgtgctgtgcagggaactacagtgcggcactgtggtttccctcctggggggagctcactttggagaaggaagtggacagatctgggctgaagaattccagtgtgaggggcacgagtcccacctttcactctgcccagtagcaccccgccctgacgggacatgtagccacagcagggacgtcggcgtagtctgctcaa

SEQ ID NO: 2 - DNA, 315 bp, artificial sequence
  the eleventh exon of human CD163L1 gene
cccacaggcagcccaggctggttggagctgatatgccctgctctggacgtgttgaagtgaaacatgcagacacatggcgctctgtctgtgattctgatttctctcttcatgctgccaatgtgctgtgcagagaattaaactgtggagatgccatatctctttctgtgggagatcactttggaaaagggaatggtctaacttgggccgaaaagttccagtgtgaagggagtgaaactcaccttgcattatgccccattgttcaacatccggaagacacttgtatccacagcagagaagttggagttgtctgttccc

SEQ ID NO: 3 - DNA, 999 bp, artificial sequence
  homologous right arm of the seventh exon of porcine CD163 gene
gtgagacccagggaatgtgttcactttgttcccatgccatgaagagggtagggttaggtagtcacagacatctttttaaagccctgtctccttccaggatacacacaaatccgcttggtgaatggcaagaccccatgtgaaggaagagtggagctcaacattcttgggtcctgggggtccctctgcaactctcactgggacatggaagatgcccatgttttatgccagcagcttaaatgtggagttgccctttctatcccgggaggagcaccttttgggaaaggaagtgagcaggtctggaggcacatgtttcactgcactgggactgagaagcacatgggagattgttccgtcactgctctgggcgcatcactctgttcttcagggcaagtggcctctgtaatctgctcaggtaagagaataagggcagccagtgatgagccactcatgacggtgccttaagagtgggtgtacctaggagttcccattgtggctcagtggtaacaaactcgactggtatccatgagggtatgggtttgatccctggccttgctcaatgggttaaggatccagcattgctgtgagctgtggtataggttgcagactctgctcaggtcccatgttgctgtgattgtggtgtaggctgactgttgcagcttcaatttgacccctagcccgggaatttccataggccacacgtgcagcactaaggaaggaaaaaaaaaaaaaaaaaaaaaagagtgggtgtgcctatagtgaagaacagatgtaaaagggaagtgaaagggattcccccattctgagggattgtgagaagtgtgccagaatattaacttcatttgacttgttacagggaaagtaaacttgactttcacggacctcctagttacctggtgcttactatatgtcttctcagagtacctgattcattcccagcctggttgacccatccccctatctctatggctatgtttatccagagcacatctatctaacact

SEQ ID NO: 4 - DNA, 6392 bp, artificial sequence
  homologous left arm of the seventh exon of porcine CD163 gene
ccttactgacaatctgagccacttcagtcagataaagaaatttcttcattaagcagagtattcacatggtttaaacttcaaaatattaaagtgtaaactctttccccaccactgtccccagctcaccaactctacttaccacagacaactgatgtggttagggtatttaaatagtaaatccaagaaaatataaacaaatccgtatatataggtttcaccccattttattatcctaatgttgcatatcatataaactatactgtcccttgggtattcacttagtaaaatattttgatcataatttcctatcagtatttaaagagctttctgaaattatttctgtataacatttcttttctcatcatctattatgtgcatttatttatattttaacttcttttattagatgaaattatcttctgcttcagcttttttttttttttaagaacacacagttgggttttttaaggttaataccacctttgttttctaagtcattaaatttgtttttctattaattcacttctgattctttgaagtttgatttctttttagcttttaacttcttgagttgtatgcttaattaattttgattctttcctatttattaatatacatatttgaagctataggttttccactgagtataccagtagctatatcgtataattgatgaactgatcctctgtgagtctgggacataaacgtcctatgactgttatgtggtagctgtgaattgctctttttagattataaagttctcatcttttatagttgaacaatttttgtcctgaatcaaatttgttggatattaatatcacatctattgctttatttattttctattctcacttttaacctctgtgaataatttcactctaggtgcctcacttttttcataatagaattgggatttatttttaaaaggactctgattaagtaattttctttttctgatatgggagatatatttgaccttaacttagtcacattatgcattgttctcttgtcatgttatgtatacataacatttattgtcattatggtacaactaaaaacatatttcactctgtgacctttatggggactcagcatttgtttaggaatgtggaagtatatttgtatatctgataatttccttccaaatttaaaaaggtttgtatattttcatattaacatatttcatattaattagcatgaatttcagctgcattaaaaggaaaaccacctgagtggtaaagaaaaagtttttttttctcttttttttttttttttttttaatggccacatctgtggcatgtgaagttcccaggctaggggtcgaataggagctacagctgccagcttgcaccacagccacaacaatgccagagccaagcctcatctgcgacctataccacaactcatggcaatgctggttccttaaccccctgagtgaggcctggggtcaaacccacatcctcatggatactaaccggctttgttaccgctgagccatgagggaaactccctttttctcattgaaaataagtcaaatagataagcagcttaaggctgtttgggtgattctgtggtccagtaattatcaaatcctactggacaagaatagagaatgtgcaaatgagggaacgtgttggtgagatcaggctctgcccactgagctatcctctgtcatgggccctgtgctgttctcagagctgtacttcctagggcattgttctcatttcaattctgagttcagtgtggagagtatacgtgtgtgggggctgcacgcttttcacaacccactttctgctgatactgatttagggatccttggattgctttacagttgagtcatcattaactagtgtcacttgccttcaaagtcagcaaaataattgtctccaaactagtaggcttctagtgtatttgctttaatccaatgccatgtgaaagtaacatggtcaaagaataagttatataccttgacctaccctgtgaccaggctcttcctcttaatttattgaccactgccttaaggtcatttgaaaccatgggtttgggaggaaggcaaggcctaaatcccgtctttgttggaaggctcactgtccttgtctttagagcatcatttttttttaaactggggtacagtttatttacagtgttgtgtcaatttctgctgtacagcatagtgacccagtcatacacatacatacattctttttctcatactatcttcaattttattttgtgctaagtctgccattttatcatcacctcagtttgaaggacaggatatttagagtttgttttttttttccccccaatcctgcaatttctaaattataagactctcaattagccgtatataacagctgcaggcacaggatgtctccctcacaaaattggtatttttccttccatttcttcttgcagtttggctatttcttgtctgagttcatctctctttttaagtgttaaaaagggcaaggaggattcatgctatgtcaacattatgattttttcttttctatacttgataagagtatacttttcccaaatgtcatccaacttttcagcatcagtttggacatggttttcttttcaaggtggtatttctctaatgtcacttgaataacaagactcgttagttctccaggctacaatatcctagtctgagtatattctgcatgttaattctattcagccacatccataatttaggttttattcctggaacacctcacttttttttttttttttggtctttttatagccataaccatggcatatggaggttcccaggctaggggtctaatctgagctttagccactggcccatgccacagccacagccatgccacatctgagccacatctgtgaccttttccacagctcacagaaacaccagatccctaacccactgagtgaggccaggggtcaaacctgtaacctcatggttcctagtcagattcgtttcctctgtaccacgatgggaattcctaatacctcacttatgataacacattctgaattatttaggattctattatactgcatgtaatagaaatcccaaatagcaaaatttgcaacttaaggcaggttcctgtctttacaaaatcatgttttcctttgctatatgtgcactttgctttcctctgtgaattcccttttttgttatatttctatagcttttggaaacacttttacttatttgggggggcctagatttttaaccctctccttgtttttctagaaatagagtttataattttatttcttcatttacttgatactttcaagagatttccaggaaaaaaattatggaaatactgtctctgtgcctgccaagttcaaactaagaattgtataatctgttttaattcttaagcatttatagatgacaaggctttgtgtctgataggggccagcgaactcagtaaagagggaagatgagaaagataatggcaagaatttatccctgaagtgtagttttgacaaaccagtcacaaagaggtctaagaaattttggtcacaaagttgttttgaatcccaggcattttatttgcaatgattgcatatgttctggaaaggacatctgaacctaagaaatagttcatttgcattgtgttatattttactaaggtctgagaaataatcttgagatgagaatgaactctacttcttcagagtctggaaggaataaattatgaaaatgtattaatgcttctttaaaccatattgtatatttatctattactaaacaaaaagaagtagctctatttatttatttatttatttatttatttatgtcttttgtctctttagggccacacctgtggcatatggaggttcccaggctagaggtccaattggagatgtagcagccagcctatgccagagccaccgcaacacgggatctgagccacgtctgtgacttacaccacagctcacagcaacgcctgatcctcaacccactgagcgaggccagggatcgaacccatgtcctcatggatgctagttgggttcattaactgctgagccatgatgggaactccaaattaattatttcttatatttgttcttcatatattcatttctatagaaagaaataaatacagattcagttaatgatggcaggtaaaagcttaacttattaatcaaaggagttaatccaggcacaaaaattcaattcatggctctctgttaaaatttaggtataggtttagcaggaagaaaaggttagtagatgcagactattacatttagaatggatggacaatgaagtcctactatacagcacagggaactatatccaatctcttgggatagaatatgatggaagacaaaatcagaacaagagagtatatatatatgtgtgtgtgtgtgtgtgtgtgtgtgtgtgtgtgtgtgtgtgtgtgactgggtcaccctgcggcacagcagaaattggcagaacattgtaaatcaactatactttaataggaaaaatacttttaagggctaaatttccaatattctaaccatgtacacagagtaaatgtcataaggatgccagtctgtgtagagattgatgtgttactagcagattcatgaaataaaggctgaggatgtagtccccaagtcacttctgagtggaagaatttctcctttgtcctggactcaaatattttaggataaaggaaaaaagaagatatttatagaagggacttgttttcaagtacttgacaaaatttcaccattaaagagaaatttgtgggagttcccatcgtggctcagtggaaacaaatccaactaggaaccatgaggttgtgggtttgatccctggcctcactcagtgggttaaggatccggtgttgccgtgagctgtggtgtaggttgcagacacggttctgatcctgcgttgctgtggctgtggctgtggtgtaggccagcagcaaacagctctgattagacccctagcctggaaacctccatatgccacaggtgcagccctaaaaagacaaaaaaagagaaaagacaaaaaaaaaaaaaaaaaaaaaaaaaaaaaaaaaaaaaaaaaaaaaaaaaaaaaaaaaagaaccaccagaggtatttatttgtttttgccttttttcactgactgttttttgtttgtttgtttgagactgatctagaagactagagattacaagaaatatggatttggctcactctaagaaactgctttcattccaaggtttgggtctatccaaaagtggaatagaatcatatgaatactagtttatgagtatttagtgagaggaatttcaagctcaaataatgattcagcaagattaaattaaggagggaattttccttgtggctgagtgggttaaggacccaatgttgtctctgtgaggatgtaggttccatcctgggctttgctcattaggttaaggatctggcattgctgcagctcagacccagtgctgccctggttgtggcttaggccaaagctgcagctccaattcaatctctggcctgggaacctccatgtgctacaaggtgcggccttaaaaggaaaaaaaaaaaaattaaatcaaggactcaagagtctttcattatttgtgttgtggaagctatatttgttttaaagtcttagttgtgtttagaaagcaagatgttcttcaactcaaatttgggagggaacttgtttcatacatttttaatggataagtggcaaaattttcatgctgaggtgatctatagtgttgtaatgcagaatatagtcagatcttgaacattttaggaagttggtgagggccaattgtgtatctgtgccatgctgataagaatgtcaagggatcacaagaattcgtgttatttgacagcagtcatctttaaaaggcatttgagaaagtccaatttcaaatgcatttcctttctttaaaagataaattgaagaaaataagtctttatttcccaagtaaattgaattgcctctcagtctgttaaaagaaactcttaccttgatgattgcgctcttaacctggcaaagattgtctttaaaatctgagctccatgtcttctgctttatttctggtgtgcctttgactccagattacagtaaatggaggactgagtatagggctaaaaagtagagagaatggatgcatattatctgtggtctccaatgtgatgaatgaagtaggcaaatactcaaaggaaagagaaagcatgctccaagaattatgggttccagaaggcaaagtcccagaattgtctccagggaaggacagggaggtctagaatcggctaagcccactgtaggcagaaaaaccaagaggcatgaatggcttccctttctcacttttcactctctggcttactcctatcatgaaggaaaatattggaatcatattctccctcaccgaaatgctatttttcag

SEQ ID NO: 5 - DNA, 20 bp, artificial sequence
  Sus scrofa
ggaactacagtgcggcactg

SEQ ID NO: 6 - DNA, 20 bp, artificial sequence
  Sus scrofa
cagtgccgcactgtagttcc

SEQ ID NO: 7 - DNA, 20 bp, artificial sequence
  Sus scrofa
acttcaacacgaccagagca

SEQ ID NO: 8 - DNA, 20 bp, artificial sequence
  Sus scrofa
tgctctggtcgtgttgaagt

SEQ ID NO: 9 - DNA, 40 bp, artificial sequence
  Sus scrofa and Homo sapiens
aagttggagttgtctgttcccgtgagacccagggaatgtg

SEQ ID NO: 10 - DNA, 29 bp, artificial sequence
  Laboratory synthesis
tatgtcgacagtgttagatagatgtgctc

SEQ ID NO: 11 - DNA, 29 bp, artificial sequence
  Laboratory synthesis
tccccgcggccttactgacaatctgagcc

SEQ ID NO: 12 - DNA, 35 bp, artificial sequence
  Sus scrofa and Homo sapiens
agcctgggctgcctgtgggctgaaaaatagcattt

SEQ ID NO: 13 - DNA, 35 bp, artificial sequence
  Sus scrofa and Homo sapiens
aaatgctatttttcagcccacaggcagcccaggct

SEQ ID NO: 14 - DNA, 40 bp, artificial sequence
  Sus scrofa and Homo sapiens
cacattccctgggtctcacgggaacagacaactccaactt

SEQ ID NO: 15 - DNA, 20 bp, artificial sequence
  Sus scrofa
gattgcgctcttaacctggc

SEQ ID NO: 16 - DNA, 20 bp, artificial sequence
  Sus scrofa
aaccctaccctcttcatggc

SEQ ID NO: 17 - DNA, 20 bp, artificial sequence
  Sus scrofa
ttctccctcaccgaaatgct

SEQ ID NO: 18 - DNA, 20 bp, artificial sequence
  Sus scrofa
gcagtgacggaacaatctcc

SEQ ID NO: 19 - DNA, 20 bp, artificial sequence
  Homo sapiens
agatgccatatctctttctg

SEQ ID NO: 20 - DNA, 20 bp, artificial sequence
  Sus scrofa
atatcggagatacccacagt

SEQ ID NO: 21 - DNA, 25 bp, artificial sequence
  Sus scrofa
ctaaccagtggctttacaccaggca

SEQ ID NO: 22 - DNA, 26 bp, artificial sequence
  Homo sapiens
cccacagaaagagatatggcatctcc

SEQ ID NO: 23 - DNA, 20 bp, artificial sequence
  Sus scrofa
gatgtccaactgctgtcact

SEQ ID NO: 24 - DNA, 19 bp, artificial sequence
  Sus scrofa
atttccacctccactgtcc

(19) United States
(12) Patent Application Publication
(10) Pub. No.: US 2019/0284580 A1
(43) Pub. Date: Sep. 19, 2019

(54) PREPARATION METHOD FOR ANTI-PORCINE REPRODUCTIVE AND RESPIRATORY SYNDROME CLONED PIG

(71) Applicant: China Agricultural University, Beijing (CN)

(72) Inventors: Ning LI, Beijing (CN); Xiaoxiang HU, Beijing (CN); Jingyao CHEN, Beijing (CN)

(21) Appl. No.: 15/542,330

(22) PCT Filed: Dec. 30, 2015
(86) PCT No.: PCT/CN2015/099835

§ 371 Date: Jul. 7, 2017

(30) Foreign Application Priority Data
Jan. 8, 2015 (CN) 201510009543.4

(51) Int. Cl. C12N 15/877; C12N 15/85; C12N 15/11; C12N 9/22; A01K 67/027; A61D 19/04

(52) U.S. Cl. CPC C12N 15/8778; C12N 15/85; C12N 15/11; C12N 9/22; A01K 67/0273; A01K 2267/0337; A61D 19/04; C12N 2310/20; C12N 2800/80; A01K 2217/05; A01K 67/0275

(57) ABSTRACT

The present invention provides a preparation method for an anti-porcine reproductive and respiratory syndrome cloned pig, comprising: transferring CRISPR/Cas9 targeting vectors and CD163 gene homologous recombination modification vectors into fibroblasts of a pig to obtain positive clone cells, a seventh exon of porcine endogenous CD163 gene being replaced with a eleventh exon of human CD163-L1 gene, so that the positive clone cells are incapable of mediating invasions of PRRSV; taking the positive cell as nuclear transfer donor cells and oocytes as nuclear transfer recipient cells to obtain a cloned embryo by adopting a somatic cell nuclear transfer technology; and impregenating a pig by transferring the cloned embryo into the uterus of the pig, to obtain a cloned pig.

### TECHNICAL FIELD

The present invention relates to the field of animal genetic engineering and genetic modification, specifically, to a modification method for the CD163 gene of an anti-porcine reproductive and respiratory syndrome cloned pig by adopting a CRISPR/Cas9 system, and a preparation method for an anti-porcine reproductive and respiratory syndrome cloned pig.

### BACKGROUND ART

Porcine reproductive and respiratory syndrome (PRRS), also known as blue ear disease, is an infectious disease caused by porcine reproductive and respiratory syndrome virus (PRRSV) and characterized by reproductive disorders in pregnant sows and respiratory symptoms in pigs at different ages, and it causes severe immunosuppression. The disease first appeared in the United States in 1987, followed by the outbreak in Europe in 1989, and gradually spread to the rest of the world. The frequent outbreaks of PRRS worldwide have caused huge economic losses, for example the “unknown swine fever” caused by PRRSV mutants in China and Viet Nam, has inflicted heavy losses on the pig industry in the two countries.

PRRSV mainly infects porcine alveolar macrophages (PAM) in the body, as well as peripheral blood mononuclear cells and sperm cells. In vitro, it is now possible to infect African green monkey kidney cell line MA104 and cell line MARC-145 derived therefrom. It has been found that, there are three receptors for PRRSV on PAM, i.e. heparin sulphate (HS), sialoadhesin (Sn) and CD163 (cluster of differentiation 163) molecules. PRRSV is first contacted with HS on the surface of PAM and then converted to perform more stable interaction with Sn. After Sn is adhered with the virus, the virus-receptor complex undergoes endocytosis mediated by the clathrin. The virus enters the early inclusion body immediately after being endocytosed, and the genome of the virus is released into the cytoplasm. This process relies on the acidification of inclusion bodies and CD163, and cathepsin E and a trypsin-like serine protease that has not yet been identified also play a role in this process.

CD163 molecule consists of nine repeating scavenger receptor cysteine rich domains (SRCR domains), two inter-domain sequences (PST I, PST II), one transmembrane sequence and one cytoplasmic tail. After CD163 was identified as a necessary receptor for infection of PAM with PRRSV, the investigators began to explore which domains of CD163 were necessary for it to act as receptors. In 2010, the Van Gorp research group prepared cells expressing mutant CD163. By detecting the ability of these cells to adhere PRRSV, it was found that SRCR5 of CD163 was necessary for infection, and meanwhile the four SRCR domains in the N-terminus and the tail in the cytoplasm were not necessary. In the same year, Phani B. Das etc. found that PRRSV envelope glycoproteins GP4 mediated the formation of envelope polyprotein complex, and acted as the ligands of CD163 molecules together with GP2a, playing an important role in the process of virus endocytosis.

CRISPR (clustered regularly interspaced short palindromic repeats)/Cas (CRISPR-associated) system is a kind of immune system that is characteristic in prokaryotic organism and directs at exogenous genetic material. It cleaves and degradates exogenous DNA, including phage and exogenous plasmids, under mediation by sequence-specific RNA. CRISPR/Cas system can be used as a site-specific gene editing system with the most important features of simple operation, low cost, and efficient action. In 2013, scientists first reported that the CRISPR/Cas system was successfully applied to cells, and subsequently applied to zebrafish, fruit flies, mice, rat, and pig rapidly. CRISPR/Cas system produces double strand break (DSB) at the target site, the cells can be repaired by non-homologous end joining (NHEJ), resulting in frameshift mutation of genes and loss of function. In addition, the system can also act together with the homologous recombination vector and oligonucleotide, so that efficient and accurate modification occurs to the target gene. In 2014, Scott J G et al. used CRISPR/Cas-mediated homologous recombination to achieve gene substitution on zebrafish. Hui Yang et al. used the same strategy to obtain a mouse with a reporter gene. CRISPR/Cas system quickly becomes an outstanding tool among the gene editing tools by virtue of its great advantages, and have been widely used in the fields such as gene function research, disease model, gene therapy and the like.

CRISPR/Cas9-mediated homologous recombination has realized precise editing of genes in zebrafish, protozoa, mice, and rats, but has not been reported for large domestic animals.

### SUMMARY OF THE INVENTION

The purpose of the present invention is to provide a preparation method for an anti-porcine reproductive and respiratory syndrome cloned pig, which uses CRISPR-Cas9 system-mediated homologous recombination to modify porcine CD163 gene so as to obtain the anti-porcine reproductive and respiratory syndrome cloned pig.

The present invention first provides the use of the seventh exon of porcine CD163 gene in the preparation of the anti-porcine reproductive and respiratory syndrome cloned pig. The nucleotide sequence of the seventh exon of porcine CD163 gene is shown in SEQ ID NO. 1.

The present invention provides a porcine CD163 gene homologous recombination modification vector in which a seventh exon of porcine CD163 gene is replaced with a eleventh exon of human CD163-L1 gene. The nucleotide sequence of the seventh exon of porcine CD163 gene is shown in SEQ ID NO. 1, and the eleventh exon of human CD163-L1 gene is shown in SEQ ID NO. 2.

The porcine CD163 gene homologous recombination modification vector according to the invention is prepared by the method comprising the following steps:

(1) fusing a eleventh exon of human CD163-L1 gene with a homologous right arm of a seventh exon of porcine CD163 gene to obtain a fusion fragment 1;

(2) fusing the fusion fragment 1 with a homologous left arm of the seventh exon of porcine CD163 gene to obtain a fusion fragment 2; and

(3) performing double enzyme digestion on the fusion fragment 2 and the vector LoxPneoLoxP2PGK with the restriction endonucleases Sal I and Sac II, respectively, and then ligating to obtain the porcine CD163 gene homologous recombination modification vector.

Wherein, the homologous right arm of the seventh exon of the porcine CD163 gene in step (1) has a nucleotide sequence as shown in SEQ ID NO. 3; and the homologous left arm of the seventh exon of porcine CD163 gene in step (2) has a nucleotide sequence as shown in SEQ ID NO. 4.

In an embodiment of the present invention, the sequence of the primer for amplifying the homologous right arm of the seventh exon of porcine CD163 gene is shown in SEQ ID NOs. 9 and 10; the sequence of the primer for amplifying the homologous left arm of the seventh exon of porcine CD163 gene is shown in SEQ ID NOs. 11 and 12; the sequence of the primer for amplifying the eleventh exon of human CD163-L1 gene is shown in SEQ ID NOs. 13 and 14.

The present invention also provides sgRNA that specifically targets the seventh exon of the porcine CD163 gene, and the sequence thereof is GGAACTACAGTGCGGCACTG (as shown in SEQ ID NO. 5). The sequence of the oligonucleotide that is complementary to and paired with the sgRNA is CAGTGCCGCACTGTAGTTCC (as shown in SEQ ID NO. 6).

The present invention also provides another sgRNA that specifically targets the seventh exon of porcine CD163 gene, and the sequence thereof is ACTTCAACACGACCAGAGCA (as shown in SEQ ID NO. 7). The sequence of the oligonucleotide that is complementary to and paired with the sgRNA is TGCTCTGGTCGTGTTGAAGT (as shown in SEQ ID NO. 8).

The present invention also provides a CRISPR/Cas9 targeting vector comprising the DNA sequence of the above-mentioned sgRNA.

The CRISPR/Cas9 targeting vector according to the present invention is prepared by a method comprising the following steps: annealing of the oligonucleotide shown in SEQ ID Nos. 5 or 6 by keeping at 94° C. for 5 min, then at 35° C. for 10 min, and then immediately placing on ice; digesting the px330 backbone vector with the restriction endonuclease Bbs I overnight, recovering, and then ligating with the annealed oligonucleotide to obtain the CRISPR/Cas9 targeting vector of the present invention.

The invention provides a preparation method for an anti-porcine reproductive and respiratory syndrome cloned pig, comprising: transferring CRISPR/Cas9 targeting vectors and porcine CD163 gene homologous recombination modification vectors together into fibroblasts of a pig to obtain positive clone cells; obtaining a cloned embryo by adopting a somatic cell nuclear transfer technology with the positive cells as nuclear transfer donor cells and oocytes as nuclear transfer recipient cells; and impregenating a pig by transferring the cloned embryo into the uterus of the pig, to obtain a CD163 gene modified anti-porcine reproductive and respiratory syndrome cloned pig.

The method for transferring CRISPR/Cas9 targeting vectors and the porcine CD163 gene homologous recombination modification vectors into fibroblasts of a pig is described as follows: the total mass of the CRISPR/Cas9 targeting vectors and the porcine CD163 gene homologous recombination modification vectors is 4 to 6μg, and they are mixed at a molar ratio of 1:1, and then transferred into about 1×10⁶ fibroblasts of pig by adopting electroporation or liposome transfection.

The invention first uses the CRISPR/Cas9 technology to mediate homologous recombination in large animals, allowing the endogenous CD163 gene to be precisely edited (see FIG. 1). The method has low cost, greatly shortens the time for obtaining homozygous pig, and ensures that the expression of CD163 will not be influenced by gene editing, which lays the foundation for the study of gene function and the establishment of disease model for large animals using CRISPR/Cas9 technology-mediated homologous recombination.

### DESCRIPTION OF DRAWINGS

FIG. 1 is a schematic view showing replacement of a seventh exon of the porcine endogenous CD163 gene with a eleventh exon of the human CD163-L1 gene as described in Example 1 of the present invention.

FIG. 2 is a flow diagram showing the construction of a porcine CD163 gene-modified vector as described in Example 1 of the present invention.

FIG. 3 is a diagram of the porcine CD163 gene-modified vector obtained in Example 1 of the present invention.

FIG. 4 shows the T7E1 enzyme digestion method for the identification of the genome cleavage by the px330 plasmid on porcine embryonic fibroblasts as described in Example 2 of the present invention, wherein 501 and 502 represent two px330 plasmids obtained in the present invention, respectively.

FIG. 5 shows the identification of cell monoclone by PCR as described in Example 3 of the present invention, wherein FIGS. 5a, 5b and 5c represent steps 1, 2 and 3 of the process, respectively. In the FIGS. 1, 2, 3, 4 and 5 represent the positive monoclonal cells numbered as No. 1, No. 2, No. 3, No. 4, and No. 5, respectively.

FIG. 6 shows the identification of newborn pig by PCR as described in Example 4 of the present invention, wherein FIGS. 6a, 6b and 6c represent steps 1, 2 and 3 of the process, respectively.

FIG. 7 is a peak figure for the identification of the homozygous condition of the newborn cloned pig by sequencing method as described in Example 4 of the present invention.

FIG. 8 shows the transcription of the modified CD163 gene in various tissues of newborn cloned pigs, which is identified by qRT-PCR, as described in Example 4 of the present invention.

FIG. 9 shows the expression of the modified CD163 gene in various tissues of the newborn cloned pigs, which is identified by Western blot, as described in Example 4 of the present invention.

FIG. 10 shows the detection on the infection of alveolar macrophages of the cloned pig and wild-type pig after challenge at different doses, as described in Example 5 of the present invention, wherein FIG. 10a is a cytopathic image after the challenge of a WUH3 strain; FIG. 10b is a cytopathic image after the challenge of a JXwn06 strain; FIG. 10c and FIG. 10d are the relative expression of the virus RNA and the titer of the virus in the supernatant after the challenge of a JXwn06 strain, respectively; and FIG. 10e and FIG. 10f are the relative expression of the virus RNA and the titer of the virus in the supernatant after the challenge of a WUH3 strain, respectively.

FIG. 11 shows the detection on the infection of alveolar macrophages of the cloned pig and wild-type pig at different time points after challenge at the same dose, as described in Example 5 of the present invention, wherein FIGS. 11a, 11b and 11c are the relative expression of the virus RNA, the titer of the virus in the supernatant, and the expression of viral protein after the challenge of a JXwn06 strain, respectively; and FIG. 11d is the relative expression of viral RNA after the challenge of a WUH3 strain.

### DETAILED DESCRIPTION OF THE PRESENT APPLICATION

The following examples are intended to illustrate the invention, but are not intended to limit the scope of the invention. Unless otherwise specified, the technical means used in the examples are conventional means known to a person skilled in the art and the used raw materials are commercially available.

Px330 vector and LoxPneoLoxP2PGK vector were purchased from Addgene Company; T4 DNA ligase and restriction endonuclease were purchased from Dalian TaKaRa Company; primer synthesis and sequencing were completed by Shanghai Shenggong and Shenzhen Huada; KOD DNA polymerase was purchased from Shanghai Dongyangfang Company; LongAmp Taq DNA polymerase, Q5 DNA polymerase and T7E1 enzyme were purchased from NEB Company; Trizol Reagent was purchased from Kangwei Century Company; reverse transcriptase was purchased from Promega Company; SYBR Green was purchased from Roche Company; EndoFree Plasmid Maxi Kit and Genome extraction kit were purchased from QIAGEN Company; and for the detailed routine experimental procedures such as digestion, ligation, recovery, transformation, and PCR amplification, please refer to “Molecular Cloning (Third Edition)”.

### EXAMPLE 1

### Construction of Porcine CD163 Gene-Modified Vector

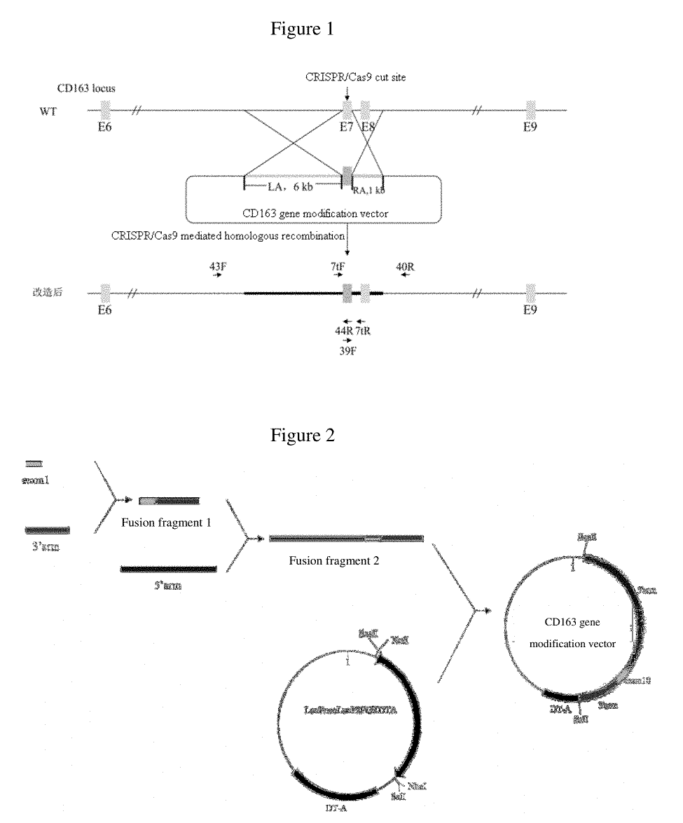

As shown in FIG. 2, the vector construction process is divided into three steps:

step 1, a eleventh exon of human CD163-L1 gene (the nucleotide sequence is shown in SEQ ID NO. 2) was fused with a homologous right arm. The eleventh exon of human CD163L1 was amplified using genomic DNA of human cells as a template, and the primers used for amplification were CD163L 1-F5′-AAATGCTATTTTTCAGCCCACAGGCAGCCCAGGCT-3′ and CD163L 1-R5′-CACATTCCCTGGGTCTCACGGGAACAGACA ACTCCAACTT-3′. The PCR product was purified and verified to be correct by sequencing. A 999 bp homologous right arm (the nucleotide sequence is shown in SEQ ID NO: 3) was obtained by amplification using the genomic DNA of a pig as a template, and the primers were RIGHT-F5′-AAGTTGGAGTTGTCTGTTCCCGTG AGACCCAGGGAATGTG-3′ and RIGHT-R 5′-TATGTCGACAGTGTT AGATAGATGTGCTC-3′, wherein the underlined was the Sal I digestion site. Sequencing was carried out to verify the product was correct. A eleventh exon of human CD163-L1 gene was fused with a homologous right arm, and the product was named fusion fragment 1, and verified to be correct by sequencing.

step 2: A 6392 bp homologous left arm (the nucleotide sequence is shown in SEQ ID NO. 4) was obtained by amplification using genomic DNA of pig cells as a template, and the primers were

Left-F
5′-TCCCCGCGGCCTTACTGACAATCTGAGCC-3′

and Left-R5′-AGCCTGGGCTGCCTGTGGGCTGAAAAATAGCATTT-3′, wherein the underlined was the Sac II digestion site. Sequencing was performed to verify the product was correct. The fusion fragment 1 was fused with the homologous left arm, and the product was named fusion fragment 2, and verified to be correct by sequencing.

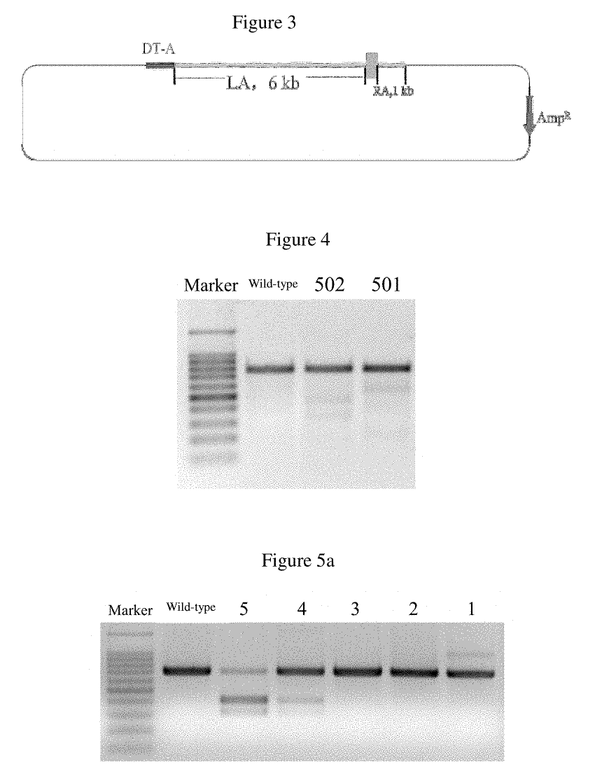

step 3: The vector LoxPneoLoxP2PGK was digested with Sal I and Sac II restriction endonucleases at 37° C., and larger fragments were recovered. The recovered fragments and the digested (Sal I and Sac II) fusion fragment 2 were ligated at 16° C. by using T4 DNA ligase to give the final porcine CD163 gene-modified vector, as shown in FIG. 3.

### EXAMPLE 2

### Construction of CRISPR-Cas9 Targeting Vector

1. The targeting site of the seventh exon of the porcine CD163 gene was predicted by using Zhang Feng laboratory website (http://crispr.genome-engineering.org/).

According to the scores in the self-assessment and prediction results, two targeting sites were selected from the candidate targeting sites, and named 501 and 502, and their sgRNA sequences were GGAACTACAGTGCGGCACTG and ACTTCAACACGACCAGAGCA, respectively. Complementary and paired oligonucleotides were synthesized according to the sgRNA sequence, as shown in Table 1, wherein the lowercase letters indicate the digestion sites.

TABLE 1
oligonucleotides sequence

| Name | Sequence (5′-3′) |
|---|---|
| px330-501F | caccgGGAACTACAGTGCGGCACTG |
| | (SEQ ID NO. 5) |
| px330-501R | aaacCAGTGCCGCACTGTAGTTCCc |
| | (SEQ ID NO. 6) |
| px330-502F | caccgACTTCAACACGACCAGAGCA |
| | (SEQ ID NO. 7) |
| px330-502R | aaacTGCTCTGGTCGTGTTGAAGTc |
| | (SEQ ID NO. 8) |

2. Two targeting vectors were constructed, and named px330-501 and px330-502. The two pairs of oligonucleotides shown in Table 1 were used, respectively, and the construction process was as follows: annealing of the oligonucleotides by keeping at 94° C. for 5 min, then at 35° C. for 10 min, and then immediately placing on ice. Px330 backbone vector was digested with restriction endonuclease Bbs I overnight and recovered, and then ligated with the annealed oligonucleotide at 16° C. for 3 h. Transformation was carried out by conventional transformation method, followed by plating. After formation of a single colony, several colonies were selected for expanding culture and sequencing. The product was verified to be correct by sequencing, indicating that two CRISPR-Cas9 targeting vectors were successfully constructed in the invention.

3. Expanded Culture of Positive Single Colony

The specific steps included: a. initial culture, single colony was picked with a tip, added into a sterile tube containing 5 ml of LB medium (tryptone 10 g, yeast extract 5 g, and NaCl 10 g all dissolved in 1 L distilled water), and incubated at 37° C. and 220 rpm for 8 h to 12 h; b. expanded culture, the overnight culture solution was transferred to a sterile triangular flask containing 100 ml of LB medium at a volume ratio of 1:500, and incubated at 37° C. and 220 rpm for 12 h to 16 h.

4. Px330 EndoFree Plasmid Maxi Kit

The px330 plasmid was extracted according to the method provided on the EndoFree Plasmid Maxi Kit, and the extracted plasmid was used for cell transfection.

5. Cell Transfection

Electroporation was performed with Lonza Nucleofector for cell transfection. The specific procedures were as follows: a. the digested and collected porcine fibroblasts (about 1×10⁶) in one well of 6-well cell culture plate, 4 μg px330 plasmid and 100 μl of Nucleofector reagent were mixed and filled into an electric shock cup to perform electroporation with T-016 procedure; b. after the electric shock was finished, 500 μL fibroblast cell culture medium (10% FBS+DMEM) preheated to 37° C. was slowly added along the inner wall of the electric shock cup, and the cells were inoculated into one well of the 6-well cell culture plate; c. culture was carried out at 37.5° C. using fibroblast culture medium (10% FBS+DMEM) free of the screened drug in an 5% CO₂ incubator.

6. Detection of the Targeting Efficiency

The cell genome was extracted according to the method provided on the genome extraction kit (Dneasy Blood & Tissue Kit). A 785 bp fragment was obtained by amplification through PCR with KOD DNA polymerase using the extracted genome and wild-type porcine fibroblast genome as templates, and the primers were CD5t-F 5′-GATTGCGCTCTTAACCTGGC-3′ and CD5t-R 5′-AACCCTACCCTCTTCATGGC-3′. The amplification conditions were as follows: 94° C. for 2 min; 94° C. for 30 sec; 60° C. for 30 sec; 68° C. for 60 sec; and 68° C. for 7 min; for 35 cycles, observing results with 1.0% agarose electrophoresis, and then recovering the PCR product and detecting the concentration. 400 ng recovered PCR product was annealed with programmed temperature drop from 95° C. to 4° C. The annealed product was digested with T7E1 enzyme at 37° C. for 1 h, and the system comprised 10 μl of annealed product, 2 μl of NEB buffer2, 0.5 μl of T7E1 and ddH₂O complementary to 20 μl. After digestion, the result was observed by polyacrylamide gel electrophoresis (PAGE), and the result showed both px330 plasmids were able to play the role of cleaving genome on porcine fibroblasts, as shown in FIG. 4.

### EXAMPLE 3

### Screening and Identification of Positive Monoclonal Cells

1. Screening of Positive Monoclonal Cells

Porcine fibroblasts (about 1×10⁶) in one well of a 6-well cell culture plate were digested and collected, and the targeting vector px330-501 constructed in Example 2 and the vector with the porcine CD163 gene modified by homologous recombination constructed in Example 1 were mixed at a molar ratio of 1:1. A total mass of 4μg was taken out, subjected to transfection according to the method in Step 5 of Example 2, then placed in a CO₂ incubator and cultured at 37.5° C. 48 h later, the cell convergence reached 80-90%, and at the moment the cells in one well were equally divided into eight 10 cm dishes. 24 h later, the cells were adhered and the culture medium was replaced with G418-containing (600 μg/mL) fibroblast culture medium (10% FBS+DMEM). The culture medium was changed every 3 to 4 days, and the culture medium was still the G418-containing (600 μg/mL) fibroblast culture medium. After cell culture for 6 to 9 days, the formation of cell cloning points can be observed. The resistant cell cloning points were found with a microscope, and marked with Marker pen. The culture medium was discarded, and the culture was washed with PBS solution once. The resistant cell cloning point was covered with cell cloning rings, and added with 10 to 30 μL 0.25% trypsin digestion solution preheated at 37° C. The cells were digested at 37.5° C. for 2 min, then the cell culture medium was added to terminate the digestion reaction, and the digested cells were inoculated into a 48-well cell culture plate for culture. When the cell convergence reached 90%, the cells were digested and inoculated into a 12-well cell culture plate for further culture. The undigested cells in the original 48-well cell culture plate were continuously cultured for the extraction of the genomic DNA of the cells. Expanded culture of the cells was continued in a 6-well cell culture plate, and the resistant cells were cryopreserved according to the cryopreservation method of porcine embryonic fibroblasts.

2. Identification of Positive Monoclonal Cells

The selected 54 monoclonal cells were identified by the following steps:

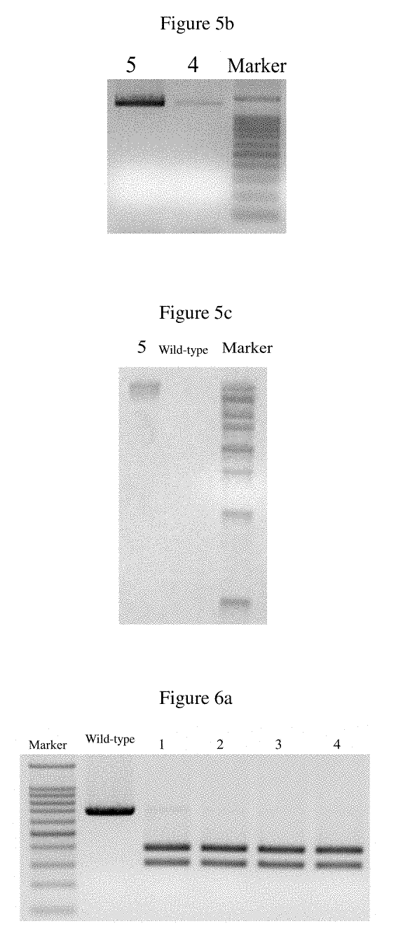

step 1: a 703 bp fragment was obtained by amplification through PCR with KOD DNA polymerase using the extracted monoclonal cells genome DNA as a template, the primers were CD7t-F(5′-TTCTCCCTCACCGAAATGCT-3′) and CD7t-R(5′-GCAGTGACGGAACAATCTCC-3′), and the PCR reaction with the wild-type porcine fibroblast genomic DNA as a template was a negative control. The amplification conditions were as follows: 94° C. for 2 min; 94° C. for 30 sec; 60° C. for 30 sec; 68° C. for 60 sec; and 68° C. for 7 min; total 35 cycles. After amplification, the result was observed with 1.0% agarose electrophoresis. Gel extraction was carried out to obtain the PCR product and the concentration thereof was detected. The purified PCR products were digested with Bbs I restriction endonucleases at 37° C. for 4 h. The results were observed by 1.5% agarose electrophoresis, as shown in FIG. 5a. Since the eleventh exon of human CD163L1 contains a digestion site of Bbs I, and the seventh exon of porcine CD163 does not contain the digestion site, Bbs I can cleave the PCR product of 703 bp into two fragments if recombination occurs.

Step 2: the monoclonal cells identified to be positive in step 1 were subjected to a second step of identification. A 1317 bp fragment was obtained by amplification through PCR with Q5 DNA polymerase, the primers were 39-F(5′-AGATGCCATATCTCTTTCTG-3′) and 40-R(5′-ATATCGGAGATAC CCACAGT-3′), and the PCR reaction with the wild-type porcine fibroblast genomic DNA as a template was a negative control. The amplification conditions were as follows: 98° C. for 30 sec; 98° C. for 10 sec; 64° C. for 30 sec; 72° C. for 45 sec; 72° C. for 2 min; total 35 cycles. After amplification, the results were observed with 1.0% agarose electrophoresis, as shown in FIG. 5b. The upstream primer 39-F used in this identification step was located on the eleventh exon of human CD163L1, and the downstream primer 40-R was located downstream of the homologous right arm. Therefore, 1317 bp fragment can be obtained by amplification if homologous recombination occurs, and cannot be obtained if the recombination does not occur.

Step 3: the monoclonal cells identified to be positive in step 2 was subjected to a third step of identification. A 6897 bp fragment was obtained by amplification through PCR with LongAmp Taq DNA polymerase, the primers were 43-F(5′-CTAACCAGTGGCTTTACACCAGGCA-3′) and 44-R(5′-CCCACAGAAAGAGATATGGCATCTCC-3′), and the PCR reaction with the wild-type porcine fibroblast genomic DNA as a template was a negative control. The amplification conditions were as follows: 94° C. for 30 sec; 94° C. for 30 sec; 60° C. for 30 sec; 65° C. for 16 min; 65° C. for 10 min; total 35 cycles. After amplification, the results were observed with 0.8% agarose electrophoresis. Eight of the 54 monoclonal clones were positive monoclones, and the results were shown in FIG. 5c. The results showed five clones which are in the state suitable for nuclear transfer, wherein No. 5 clone was positive. The upstream primer 43-F used in this identification step was located upstream of the homologous left arm, and the downstream primer 44-R was located on the eleventh exon of human CD163L1. Therefore, the 6897 bp fragment can be obtained by amplification if homologous recombination occurs, and cannot be obtained if the recombination does not occur.

In this example, the results of the first identification step showed that the eleventh exon of human CD163L1 was inserted into the genome of porcine fibroblasts, and the results of the identification steps 2 and 3 showed that the insertion position was correct, and the positive monoclonal cells can be confirmed by the three identification steps.

### EXAMPLE 4

### Preparation and Identification of CD163 Gene-Modified Anti-Porcine Reproductive and Respiratory Syndrome Cloned Pig

1. Preparation of CD163 Gene-Modified Anti-Porcine Reproductive and Respiratory Syndrome Cloned Pig

The positive cells obtained in Example 3, in which homologous recombination successfully occurs, were used as nuclear transfer donor cells, and the prepubertal sow oocytes maturated in vitro were used as nuclear transfer recipient cells. The nuclear-transfer donor cells were transferred into the oocytes from which the nucleus had been removed, and then subjected to electrical fusion and activation to construct cloned embryos. Cloned embryos with good morphology were selected, and transferred by surgery into the uterus of naturally estrus multiparous sows for pregnancy. The surgical embryo transfer comprised the steps of: conducting conventional anesthesia with Shu Tai, allowing the sow to lie supine on an operating support, making a surgical incision having a length of about 8 cm on the medioventral line to expose the ovary, the fallopian tube and the uterus, allowing the the embryo transfer tube to enter along the umbrella of the fallopian tube into about 5 cm, transplanting the embryo (300 or more) into the ampulla-isthmus juncture of the fallopian tube. 30 days after the embryo transfer, it was detected whether pregnancy occurred or not by B-type ultrasonography.

2. PCR Detection of Newborn Pigs

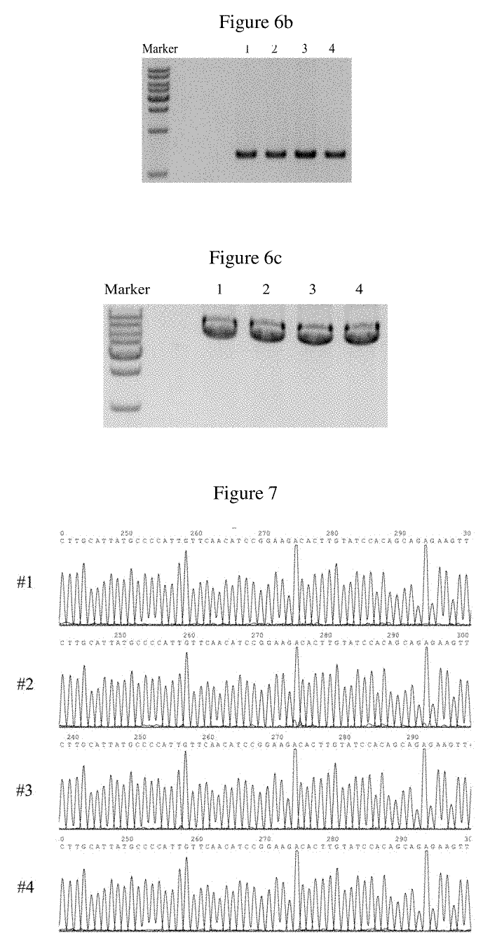

PCR was performed on the cloned pigs born after full-term pregnancy from step 1, and the detection method was consistent with the detection method of the monoclonal cells in Example 3. The results were shown in FIG. 6a, FIG. 6b and FIG. 6c. The PCR products of primers CD7tF and CD7tR were completely cleaved by Bbs I, and further sequencing revealed the peak figure was single, as shown in FIG. 7, indicating that the newborn pig was homozygous.

3. qRT-PCR Detection of Newborn Pigs

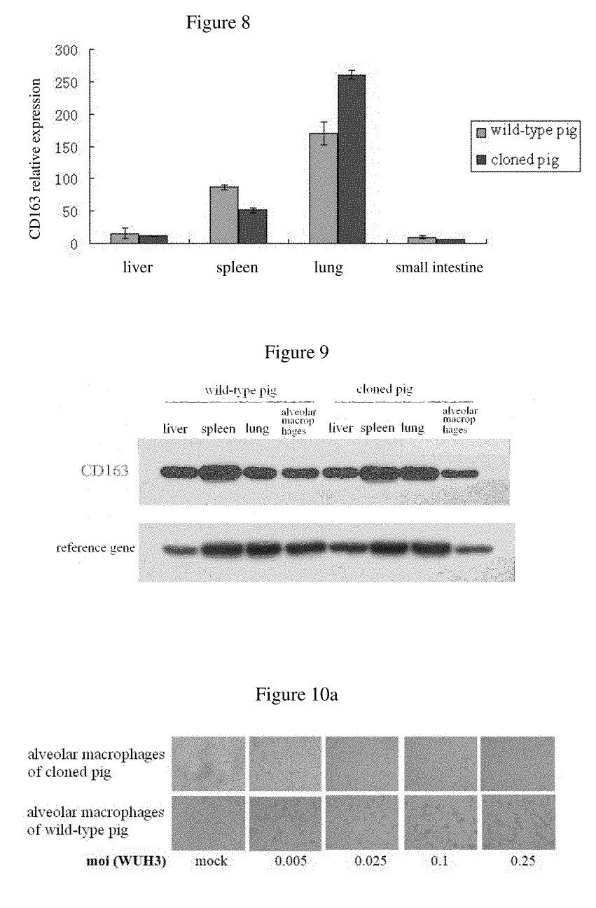

The transcription of CD163 in various tissues of cloned pigs was detected by qRT-PCR. The four tissues liver, spleen, lung and small intestine of the cloned pig and wild-type pig were taken, each 30 to 50 mg, added with 1 ml Trizol, and then homogenized with steel beads for 10 min. After homogenization was finished, the sample was added with 0.2 ml of chloroform, then homogenized for 15 sec, kept at room temperature for 2 min, and centrifuged under the conditions of 4° C. and 12000 rpm. for 15 min At this time, the sample was divided into three layers: a red organic phase, a middle layer and an upper layer of colorless water phase, wherein RNA was mainly present in the water phase. The water phase (about 600 μl) was transferred to another centrifuge tube, added with an equal volume of isopropanol, mixed upside down, kept at room temperature for 10 min, and centrifuged under the conditions of 4° C. and 12000 rpm for 15 min. The supernatant was discarded, and the precipitate was washed twice by adding 1 ml of 75% ethanol, centrifuged under the conditions of 4° C. and 12000 rpm for 3 min, and the supernatant sucked and discarded carefully (be careful not to discard the RNA precipitate), kept at room temperature for 2 min, then added with 30 μl RNase-free water to sufficiently dissolve the RNA precipitate. 2 μg RNA was taken out and subjected to reverse transcription with reverse transcriptase to obtain cDNA. The system for real-time fluorescence quantification was as follows: SYBR Green 7.5 μl, the upstream and downstream primers were 0.2 μl respectively, template cDNA and ddH2O added until the volume of the system was 15 μl. The primers were 136-F (5′-GATGTCCAACTGCTGTCACT-3′) and 137-R (5′-ATTTCCACCTCCACTGTCC-3′). The instrument used for fluorescence quantification was a Roche LightCycler 480 fluorescence quantitative PCR instrument. The results of qRT-PCR showed that the transcriptional trend of CD163 in each tissue of CD163 gene-modified newborn cloned pigs was consistent with that in wild-type pigs, as shown in FIG. 8.

4. Western Blot Detection of Cloned Pigs

The tissues liver, spleen and lung of the cloned pig were taken in an amount of 100 mg for each tissue, and the tissues liver, spleen and lung of the wild-type pig were taken as the negative control in an amount of 100 mg for each tissue. The tissues were cut into small pieces, added with 1 ml of lysis solution (the lysis solution was added with PMSF several minutes before use, so that the final concentration of PMSF was 1 mM), homogenized with a glass homogenizer until full lysis. Subsequently, centrifugation was performed at 12000 g for 3 minutes, and the supernatant was collected to obtain the total protein of each tissue. Alveolar macrophages of cloned pigs and wild-type pigs were thawed to one well of a 6-well plate, respectively, cultured for 24 h, and washed with PBS once following removal the culture medium, then added with 200 μl of lysate in each well, blown for several times, and centrifuged at 12000 g for 3 min, and the supernatant was collected to obtain the total protein of alveolar macrophages. The concentration was measured with BCA protein concentration assay kit. The total proteins were taken, each 20 μg, and subjected to electrophoresis with 6% SDS-PAGE gel at 60V for 1 h and then 90V for 2 h. After electrophoresis, membrane transfer was conducted using a Bio-Rad wet membrane transfer instrument 350 mA for 80 min. After membrane transfer was finished, the membrane was closed overnight with 5% skimmed milk powder, then incubated with Rabbit anti-pig primary antibody (1:300 dilution) for 2 h, washed with TBST for 3×10 min, then incubated with HRP labeled goat anti-rabbit secondary antibody (1: 10000 dilution) for 1 h, and washed with TBST for 3×10 min, and finally subjected to BCL color development. The results were shown in FIG. 9. The expression level of CD163 in each tissue of the anti-porcine reproductive and respiratory syndrome cloned pig of the present invention had no difference from that of the wild-type pig, indicating that the expression of CD163 of the anti-porcine reproductive and respiratory syndrome cloned pig of the present invention was not affected by the replacement of the seventh exon of the CD163 gene with the eleventh exon of the human CD163-L1 gene.

5. Cloned Pigs PAM Cell Challenge Test

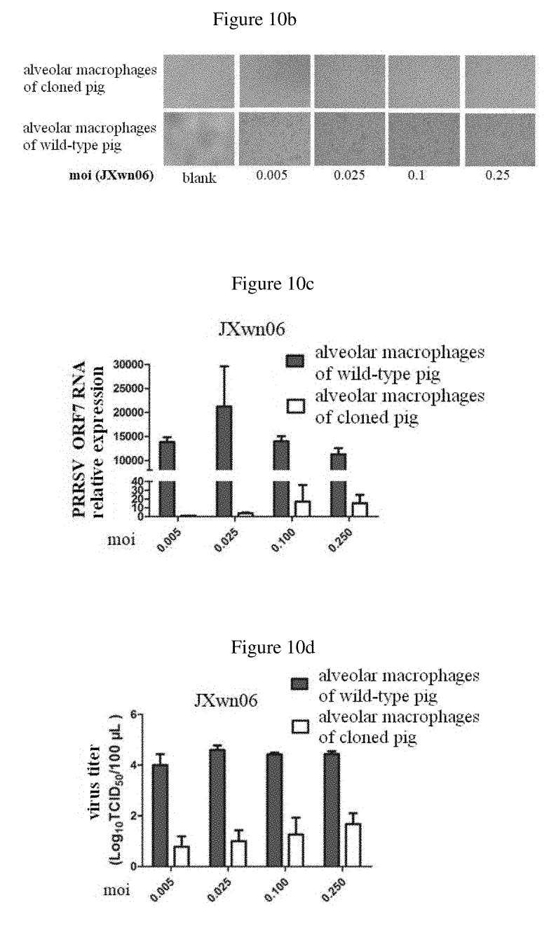

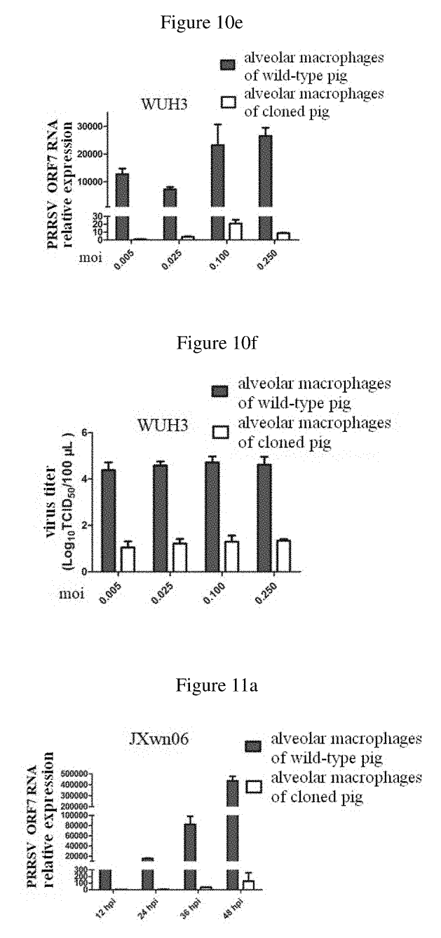

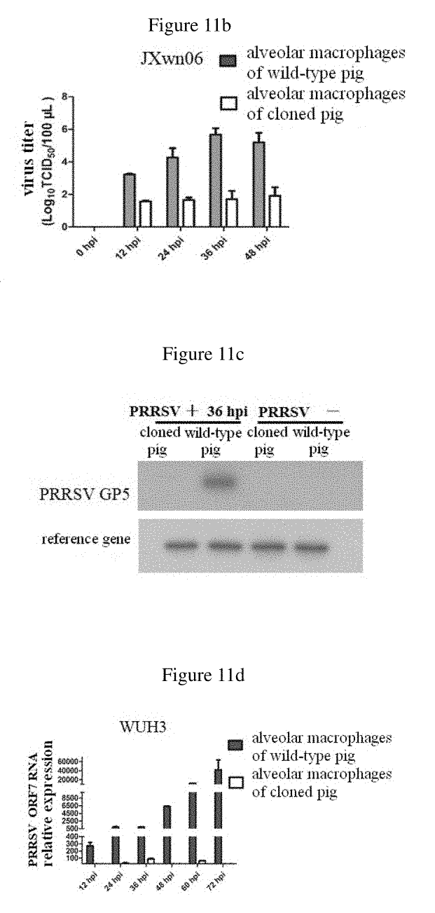

Cloned pig and SPF (Specific Pathogen Free) wild-type control pigs were dissected, the lungs were taken out in an environment as clean as possible (clamp the trachea of the pig with hemostatic forceps before the trachea was cut off), and lung lavage was carried out in an ultra-clean bench; HBSS buffer (added with penicillin and streptomycin) was slowly poured into the lungs in batches (about 500 to 800 ml each time), the filled lung was subjected to lung massage every time, and all lavage fluid was collected, centrifuged at 400 g to collect cells; the collected cells were washed with RPMI 1640 culture medium (added with double antibody) twice, and the cells were collected, counted, resuspended in cell cryoprotectant, subpackaged and cryopreserved (10⁷ to 10⁸ cells in each tube). Cells were thawed before use, and then cultured with RPMI 1640 culture medium containing 10% FBS. When virus infected the cells, a certain amount of the virus first adsorbed with the cells for 1 hour (a small amount of culture medium), and then culture medium was replenished for continue culture. Firstly, the PAM cells of cloned pig and PAM cells of wild-type pig were challenged with JXwn06 strains and WUH3 strains at different challenge doses. The cytopathic effect was observed 48 hours after challenge. The supernatant was collected, and the virus titer was measured by TCID₅₀ method. The cells were collected and extracted for RNA, and the viral load was detected by Real-Time PCR method. The results were shown in FIG. 10. The PAM cells of the anti-porcine reproductive and respiratory syndrome cloned pig obtained in the present invention had the ability to resist PRRSV at different challenge doses and reached the level of full protection. Subsequently, the cytopathic effects at different time points after challenge at the same challenge dose were observed. Similarly, the supernatant was collected, and the virus titer was measured by TCID₅₀ method; the cells were collected and extracted for RNA, and the viral load was detected by Real-Time PCR method. The results were shown in FIG. 11. The PAM cells of the anti-porcine reproductive and respiratory syndrome cloned pig obtained in the present invention had the ability to resist PRRSV at different time points after the challenge at the same dose and reached a level of full protection.

Although the invention has been described in detail by way of general description, specific embodiments and tests, it will be apparent to a person skilled in the art that modifications and improvements may be made thereto without departing from the spirit of the invention. Accordingly, these modifications or improvements that are made without departing from the spirit of the invention fall within the scope of the present invention as claimed.

### INDUSTRIAL APPLICABILITY

The invention provides a preparation method for an anti-porcine reproductive and respiratory syndrome cloned pig, in which the CRISPR-Cas9 system is used to mediate homologous recombination to modify porcine CD163 gene, thereby obtaining the anti-porcine reproductive and respiratory syndrome cloned pig. The method has a low cost, greatly shortens the time for obtaining homozygous pig, and ensures that the expression of CD163 will not be affected by gene editing, which lays the foundation for the study of gene function and the establishment of disease model for large animals using CRISPR/Cas9 technology-mediated homologous recombination.

1. (canceled)

2. A porcine CD163 gene homologous recombination modification vector, comprising a porcine CD163 gene in which a seventh exon of a porcine CD163 gene is replaced with an eleventh exon of a human CD163-L1 gene, wherein the nucleotide sequence of the seventh exon of porcine CD163 gene is shown in SEQ ID NO. 1, and the nucleotide sequence of the eleventh exon of human CD163-L1 gene is shown in SEQ ID NO. 2.

3. The A method of preparing the porcine CD163 gene homologous recombination modification vector according to claim 2, it is prepared by a method comprising:
(1) fusing a eleventh exon of a human CD163-L1 gene with a homologous right arm of a seventh exon of a porcine CD163 gene to obtain a fusion fragment 1;
(2) fusing the fusion fragment 1 obtained in step (1) with a homologous left arm of the seventh exon of the porcine CD163 gene to obtain a fusion fragment 2; and
(3) performing double enzyme digestion on the fusion fragment 2 obtained in step (2) and the vector LoxPneoLoxP2PGK with the restriction endonucleases Sal I and Sac II, respectively, and then ligating to obtain the porcine CD163 gene homologous recombination modification vector.

4. The method according to claim 3, wherein the homologous right arm of the seventh exon of the porcine CD163 gene in step (1) has a nucleotide sequence as shown in SEQ ID NO. 3; and the homologous left arm of the seventh exon of the porcine CD163 gene in step (2) has a nucleotide sequence as shown in SEQ ID NO. 4.

5. The method according to claim 3, wherein the sequences of the primers for amplifying the homologous right arm of the seventh exon of porcine CD163 gene are shown in SEQ ID NOs. 9 and 10; the sequences of the primers for amplifying the homologous left arm of the seventh exon of porcine CD163 gene are shown in SEQ ID NOs. 11 and 12; the sequences of the primers for amplifying the eleventh exon of human CD163-L1 gene are shown in SEQ ID NOs. 13 and 14.

6. A small guide ribonucleic acid (SgRNA) specifically targeting the seventh exon of porcine CD163 gene, wherein said SgRNA having the DNA sequence of SEQ ID NO. 5 or is shown in SEQ ID NO. 7.

7. A CRISPR/Cas9 targeting vector comprising the DNA sequence of the sgRNA of claim 6.

8. The CRISPR/Cas9 targeting vector according to claim 7, wherein the CRISPR/Cas9 targeting vector is prepared by a method comprising the following steps:
annealing of the oligonucleotide shown in SEQ ID Nos. 5 or 6, or the oligonucleotide shown as SEQ ID Nos. 7 and 8 by keeping at 94° C. for 5 min, then at 35° C. for 10 min, and then immediately placing on ice; and
digesting the px330 backbone vector with the restriction endonuclease Bbs I overnight, recovering, and then ligating with the annealed oligonucleotide.

9. A preparation method for an anti-porcine reproductive and respiratory syndrome cloned pig, comprising:
transferring CRISPR/Cas9 targeting vectors according to claim 7 and a porcine CD163 gene homologous recombination modification vector comprising a porcine CD163 gene in which a seventh exon of a porcine CD163 gene is replaced with an eleventh exon of a human CD163-L1 gene, in which the nucleotide sequence of the seventh exon of porcine CD163 gene is shown in SEQ ID NO. 1 and the nucleotide sequence of the eleventh exon of human CD163-L1 gene is shown in SEQ ID NO. 2, into fibroblasts of a pig to obtain positive clone cells;
obtaining a cloned embryo by adopting a somatic cell nuclear transfer technology with the positive cells as nuclear transfer donor cells and oocytes as nuclear transfer recipient cells; and
transferring the cloned embryo into the uterus of the pig for pregnancy to obtain a CD163 gene modified anti-porcine reproductive and respiratory syndrome cloned pig.

10. The preparation method according to claim 9, wherein the method for transferring CRISPR/Cas9 targeting vectors and the porcine CD163 gene homologous recombination modification vector into fibroblasts of a pig is as follows: the total mass of the CRISPR/Cas9 targeting vectors and the porcine CD163 gene homologous recombination modification vector are 4 to 6 μg, and they are mixed at a molar ratio of 1:1, and then transferred into about 1×10⁶ fibroblasts of pig by adopting electroporation or liposome transfection.

11. A preparation method for an anti-porcine reproductive and respiratory syndrome cloned pig, comprising:
transferring CRISPR/Cas9 targeting vectors comprising the DNA sequence of a small guide ribonucleic acid (SgRNA) specifically targeting the seventh exon of porcine CD163 gene, wherein said SgRNA having the DNA sequence of SEQ ID NO. 5 or SEQ ID NO. 7 and the porcine CD163 gene homologous recombination modification vector according to claim 3 into fibroblasts of a pig to obtain positive clone cells;
obtaining a cloned embryo by adopting a somatic cell nuclear transfer technology with the positive cells as nuclear transfer donor cells and oocytes as nuclear transfer recipient cells; and
transferring the cloned embryo into the uterus of the pig for pregnancy to obtain a CD163 gene modified anti-porcine reproductive and respiratory syndrome cloned pig.

12. A preparation method for an anti-porcine reproductive and respiratory syndrome cloned pig, comprising:
transferring CRISPR/Cas9 targeting vectors comprising the DNA sequence of a small guide ribonucleic acid (SgRNA) specifically targeting the seventh exon of porcine CD163 gene, wherein said SgRNA having the DNA sequence of SEQ ID NO. 5 or SEQ ID NO. 7 and the porcine CD163 gene homologous recombination modification vector according to claim 4 into fibroblasts of a pig to obtain positive clone cells;
obtaining a cloned embryo by adopting a somatic cell nuclear transfer technology with the positive cells as nuclear transfer donor cells and oocytes as nuclear transfer recipient cells; and
transferring the cloned embryo into the uterus of the pig for pregnancy to obtain a CD163 gene modified anti-porcine reproductive and respiratory syndrome cloned pig.

13. A preparation method for an anti-porcine reproductive and respiratory syndrome cloned pig, comprising:
transferring CRISPR/Cas9 targeting vectors comprising the DNA sequence of a small guide ribonucleic acid (SgRNA) specifically targeting the seventh exon of porcine CD163 gene, wherein said SgRNA having the DNA sequence of SEQ ID NO. 5 or SEQ ID NO. 7 and the porcine CD163 gene homologous recombination modification vector according to claim 5 into fibroblasts of a pig to obtain positive clone cells;
obtaining a cloned embryo by adopting a somatic cell nuclear transfer technology with the positive cells as nuclear transfer donor cells and oocytes as nuclear transfer recipient cells; and
transferring the cloned embryo into the uterus of the pig for pregnancy to obtain a CD163 gene modified anti-porcine reproductive and respiratory syndrome cloned pig.

14. A preparation method for an anti-porcine reproductive and respiratory syndrome cloned pig, comprising:
transferring CRISPR/Cas9 targeting vectors according to claim 8 and a porcine CD163 gene homologous recombination modification vector comprising a porcine CD163 gene in which a seventh exon of a porcine CD163 gene is replaced with an eleventh exon of a human CD163-L1 gene, in which the nucleotide sequence of the seventh exon of porcine CD163 gene is shown in SEQ ID NO. 1 and the nucleotide sequence of the eleventh exon of human CD163-L1 gene is shown in SEQ ID NO. 2 into fibroblasts of a pig to obtain positive clone cells;
obtaining a cloned embryo by adopting a somatic cell nuclear transfer technology with the positive cells as nuclear transfer donor cells and oocytes as nuclear transfer recipient cells; and
transferring the cloned embryo into the uterus of the pig for pregnancy to obtain a CD163 gene modified anti-porcine reproductive and respiratory syndrome cloned pig.

15. A preparation method for an anti-porcine reproductive and respiratory syndrome cloned pig, comprising:
annealing of the oligonucleotide shown in SEQ ID Nos. 5 or 6, or the oligonucleotide shown as SEQ ID Nos. 7 and 8 by keeping at 94° C. for 5 min, then at 35° C. for 10 min, and then immediately placing on ice;
digesting the px330 backbone vector with the restriction endonuclease Bbs I overnight, recovering, and then ligating with the annealed oligonucleotide to obtain CRISPR/Cas9 targeting vectors;
transferring the CRISPR/Cas9 targeting vectors comprising a DNA sequence of a small guide ribonucleic acid (SgRNA) specifically targeting the seventh exon of porcine CD163 gene into fibroblasts of a pig to obtain positive clone cells, wherein said SgRNA comprises the DNA sequence of SEQ ID NO. 5, 6, 7 or 8 and the porcine CD163 gene homologous recombination modification vector according to claim 3;
obtaining a cloned embryo by adopting a somatic cell nuclear transfer technology with the positive cells as nuclear transfer donor cells and oocytes as nuclear transfer recipient cells; and
transferring the cloned embryo into the uterus of the pig for pregnancy to obtain a CD163 gene modified anti-porcine reproductive and respiratory syndrome cloned pig.

16. A preparation method for an anti-porcine reproductive and respiratory syndrome cloned pig, comprising:
annealing of the oligonucleotide shown in SEQ ID Nos. 5 or 6, or the oligonucleotide shown as SEQ ID Nos. 7 and 8 by keeping at 94° C. for 5 min, then at 35° C. for 10 min, and then immediately placing on ice; and
digesting the px330 backbone vector with the restriction endonuclease Bbs I overnight, recovering, and then ligating with the annealed oligonucleotide to obtain CRISPR/Cas9 targeting vectors;
transferring CRISPR/Cas9 targeting vectors comprising the DNA sequence of a small guide ribonucleic acid (SgRNA) specifically targeting the seventh exon of porcine CD163 gene into fibroblasts of a pig to obtain positive clone cells, wherein said SgRNA comprises the DNA sequence of SEQ ID NO. 5, 6, 7 or 8 and the porcine CD163 gene homologous recombination modification vector according to claim 4;
obtaining a cloned embryo by adopting a somatic cell nuclear transfer technology with the positive cells as nuclear transfer donor cells and oocytes as nuclear transfer recipient cells; and
transferring the cloned embryo into the uterus of the pig for pregnancy to obtain a CD163 gene modified anti-porcine reproductive and respiratory syndrome cloned pig.

17. A preparation method for an anti-porcine reproductive and respiratory syndrome cloned pig, comprising:
annealing of the oligonucleotide shown in SEQ ID Nos. 5 or 6, or the oligonucleotide shown as SEQ ID Nos. 7 and 8 by keeping at 94° C. for 5 min, then at 35° C. for 10 min, and then immediately placing on ice; and
digesting the px330 backbone vector with the restriction endonuclease Bbs I overnight, recovering, and then ligating with the annealed oligonucleotide to obtain CRISPR/Cas9 targeting vectors;
transferring CRISPR/Cas9 targeting vectors comprising the DNA sequence of a small guide ribonucleic acid (SgRNA) specifically targeting the seventh exon of porcine CD163 gene into fibroblasts of a pig to obtain positive clone cells, wherein said SgRNA comprises the DNA sequence of SEQ ID NO. 5, 6, 7 or 8 and the porcine CD163 gene homologous recombination modification vector according to claim 5;
obtaining a cloned embryo by adopting a somatic cell nuclear transfer technology with the positive cells as nuclear transfer donor cells and oocytes as nuclear transfer recipient cells; and
transferring the cloned embryo into the uterus of the pig for pregnancy to obtain a CD163 gene modified anti-porcine reproductive and respiratory syndrome cloned pig.
